(19) United States
(12) Patent Application Publication
(10) Pub. No.: US 2022/0047530 A1
(43) Pub. Date: Feb. 17, 2022

(54) USE OF HORDENINE IN PREPARING DRUG FOR TREATING HYPOPHYSOMA

(71) Applicants: Xiong WANG, Wuhan (CN); Wuhan Third Hospital, Wuhan (CN)

(72) Inventors: Xiong WANG, Wuhan (CN); JinHu WU, Wuhan (CN)

(21) Appl. No.: 17/412,236

(22) Filed: Aug. 25, 2021

Related U.S. Application Data
(63) Continuation of application No. PCT/CN2020/070961, filed on Jan. 8, 2020.

(30) Foreign Application Priority Data
May 22, 2019 (CN) 201910432867.7

(51) Int. Cl. A61K 31/137; A61P 35/00

(52) U.S. Cl. CPC A61K 31/137; A61P 35/00

(57) ABSTRACT

The present disclosure discloses the use of hordenine in the preparation of drugs for treating pituitary tumors, and relates to the technical field of biopharmaceuticals. Specifically, in this use, the inventor has found that hordenine can help treat prolactinoma and adrenocorticotrophic hormone adenoma and exert drug efficacy by inhibiting a TLR4/NF-κB/MAPK signaling pathway, solving the dilemma of medication shortage for patients of prolactinoma and adrenocorticotrophic hormone adenoma.

### CROSS REFERENCE TO RELATED APPLICATIONS

This application is a U.S. continuation of co-pending International Patent Application Number PCT/CN2020/070961, filed on Jan. 8, 2020, which claims the benefit and priority of Chinese Patent Application No. 201910432867.7 entitled “USE OF HORDENINE IN PREPARING DRUGS FOR TREATING PITUITARY TUMORS”, filed before China National Intellectual Property Administration on May 22, 2019, the entire contents of which are incorporated herein by reference.

### TECHNICAL FIELD

The present disclosure relates to the technical field of biopharmaceuticals, in particular to the use of hordenine in the preparation of drugs for treating pituitary tumors.

### BACKGROUND

Hypophysoma is a common benign intracranial tumor. Its incidence is second only to glioma and meningioma among intracranial tumors, accounting for about 10% of intracranial tumors. Moreover, as the diagnosing level is continually improved, the incidence tends to increase year by year. The constant growth of tumor may compress the peripheral structures in sella area, such as the optic nerve, cavernous sinus, cerebral artery, hypothalamus, and the like, and may even affect the frontal lobe and brainstem, leading to severe dysfunction. Meanwhile, tumor growth may also lead to parasecretion of pituitary hormone. According to peripheral blood hormone levels, pituitary adenomas are divided into non-functional pituitary adenomas (NFPA) and functional pituitary adenomas, including: prolactinoma (PRL), adrenocorticotrophic hormone adenoma (ACTH), growth hormone adenoma (GH), thyroid stimulating hormone adenoma (TSH), pituitary gonadotropin adenoma (PGA) and mixed hormone secreting adenoma, etc.

Prolactinoma is the most common pituitary tumor, accounting for about 80% to 85% of pituitary adenomas, and it is the most common hypothalamic-pituitary disease caused by excessive secretion of prolactin (PRL) by pituitary prolactinoma. Typical clinical manifestations include: amenorrhea, galactorrhea, infertility, and hyperprolactinemia in females; impotence, hypogonadism, and breast development in males; and symptoms and signs caused by tumor compression and invasion of peripheral structures. Prolactin microadenomas with clinical symptoms generally do not grow into macroadenomas. Some adenomas have aggressive growth and exhibit adenoma enlargement. At present, during clinical treatment, about 8% to 25% of patients with pituitary prolactinoma have drug resistance after treatment with bromocriptine. Currently, bromocriptine is the only drug which is effective for the treatment of prolactinoma in China. Bromocriptine has a significant therapeutic effect, but it is expensive and has side effects. For example, bromocriptine can effectively reduce PRL, restore gonadal function, and shrink or control tumor growth for 80% to 90% of the patients. Its side effects are mainly gastrointestinal reactions; and when the dose is high, it may cause dizziness, orthostatic hypotension, headache, drowsiness and constipation, etc. There is no alternative drug for patients who are resistant to bromocriptine or intolerant to its adverse reactions.

Adrenocorticotropic hormone adenoma (ACTH-Pas), also known as Cushing's disease, is a functional pituitary adenoma accompanied by secretion of adrenocorticotropic hormone (ACTH), accounting for about 14% of all pituitary adenomas and about 70% of Cushing's syndromes. The incidence of this disease in European and American countries is 39 per million. In China, it lacks large-scale epidemiological data. Since the diagnosis of this disease is mainly based on laboratory hormone level detection, clinical symptoms and signs, imaging examinations and pathological immunohistochemistry assays of the patients, the early diagnosis of Cushing's disease is very difficult and misdiagnosis easily occurs. The patients see a doctor often because of suffering from serious complications caused by hypercortisolemia, such as hypertension, diabetes, hyperlipidemia, osteoporosis, and depression, etc. In terms of treatment, for the vast majority of the patients of Cushing's disease, transsphenoidal adenomaectomy is still the first choice in clinical treatment. Radiation therapy and drug therapy with pasireotide, ketoconazole and the like are often used as postoperative adjuvant treatments of Cushing's disease. Although it is reported that the success rate of surgical operation is 65% to 90%, due to the differences in the biological behavior of the tumors and the differences in the operation level of the operators, the tumor recurrence rate is 3% to 47%, and the average recurrence time is 16 to 49 months. The prognosis of relapsed patients is poor and the mortality rate is high. In recent years, the research on Cushing's disease at molecular level has mainly focused on the occurrence and progression of tumor, aggressiveness of tumor and hormone secretion, etc., but the pathogenesis of Cushing's disease is not fully understood yet.

Therefore, for the health of patients with pituitary prolactinoma and adrenocorticotropic hormone adenoma, and for the safety of treatment, there is an urgent need to research and develop new drugs.

### SUMMARY

The present disclosure provides the use of hordenine in the preparation of an inhibitor for inhibiting a MAPK signaling pathway.

The present disclosure provides a method for inhibiting a MAPK signaling pathway, comprising contacting hordenine or a pharmaceutically acceptable salt thereof with the MAPK signaling pathway in vivo or in vitro.

The present disclosure provides the use of hordenine or a pharmaceutically acceptable salt thereof as an inhibitor for inhibiting a MAPK signaling pathway.

In one or more embodiments, the MAPK signaling pathway is TLR4/NF-κB/MAPK signaling pathway.

In one or more embodiments, the inhibition of a MAPK signaling pathway is used for inhibiting the expression of PRL.

In one or more embodiments, the inhibition of a MAPK signaling pathway is used for inhibiting the expression of ATCH.

In one or more embodiments, the inhibition of a MAPK signaling pathway is used for inhibiting the expression of MAPK12.

In one or more embodiments, the inhibition of a MAPK signaling pathway is used for inhibiting the expression of TLR4.

In one or more embodiments, the inhibition of a MAPK signaling pathway is used for inhibiting the expression of IL-β.

In one or more embodiments, the inhibition of a MAPK signaling pathway is used for inhibiting the expression of TNF-α.

The present disclosure provides a MAPK signaling pathway inhibitor, wherein the inhibitor comprises hordenine.

In one or more embodiments, the MAPK signaling pathway is TLR4/NF-κB/MAPK signaling pathway.

In one or more embodiments, a MAPK signaling pathway inhibitor is used for inhibiting the expression of at least one of MAPK12, TLR4, PRL, ACTH, IL-β, and TNF-α.

The present disclosure provides the use of a MAPK signaling pathway inhibitor in the manufacture of a medicament for the treatment of pituitary tumors, including prolactinoma and adrenocorticotrophic hormone adenoma.

In one or more embodiments, the MAPK signaling pathway inhibitor is a MAPK signaling pathway inhibitor as described in the present disclosure.

The present disclosure provides a method for treating pituitary tumors, comprising administering a MAPK signaling pathway inhibitor to a subject in need.

The present disclosure provides the use of a MAPK signaling pathway inhibitor in the treatment of pituitary tumors.

In one or more embodiments, the pituitary tumors include prolactinoma and adrenocorticotrophic hormone adenoma.

In one or more embodiments, the MAPK signaling pathway inhibitor is a MAPK signaling pathway inhibitor as described herein.

The present disclosure provides the use of a MAPK signaling pathway inhibitor as described herein in the manufacture of a medicament for the treatment of a disease associated with MAPK signaling pathway abnormality.

In one or more embodiments, the MAPK signaling pathway abnormality is a TLR4/NF-κB/MAPK signaling pathway abnormality.

In one or more embodiments, the MAPK signaling pathway abnormality comprises the overexpression of at least one of MAPK12, TLR4, PRL, ACTH, IL-β, and TNF-α.

The present disclosure provides a method for treating a disease associated with MAPK signaling pathway abnormality, comprising administering a MAPK signaling pathway inhibitor as described in the present disclosure to a subject in need.

The present disclosure provides the use of a MAPK signaling pathway inhibitor as described herein in the treatment of a disease associated with MAPK signaling pathway abnormality.

In one or more embodiments, the MAPK signaling pathway abnormality is TLR4/NF-κB/MAPK signaling pathway abnormality.

In one or more embodiments, the MAPK signaling pathway abnormality comprises the overexpression of at least one of MAPK12, TLR4, PRL, ACTH, IL-β, and TNF-α.

### BRIEF DESCRIPTION OF THE DRAWINGS

In order to illustrate the technical solutions of the embodiments of the present disclosure more clearly, the drawings that need to be used in the embodiments are briefly introduced hereinbelow. It should be understood that the following drawings only show some of the embodiments of the present disclosure, and therefore, should not be regarded as a limitation of the scope. For those of ordinary skill in the art, they may also obtain other related drawings according to these drawings without doing inventive work.

FIG. 1 illustrates growth curves of rats in each group in Example 1 of the present disclosure;

FIG. 2 illustrates the pituitary comparison of rats in the normal group and the model group in Example 1 of the present disclosure;

FIG. 3 illustrates the expression results of PRL, CD68 and NLRP3 in rats as measured by Western Blotting in Example 1 of the present disclosure;

FIG. 4 illustrates the quality control results of BioB-BioC-BioD-Cre hybridization in Example 1 of the present disclosure;

FIG. 5 illustrates the quality control results of Pos-Neg negative and positive in Example 1 of the present disclosure;

FIG. 6 is a histogram of differential gene statistics in Example 1 of the present disclosure;

FIG. 7 illustrates the expression results of differential genes in the gene clustering diagram in Example 1 of the present disclosure;

FIG. 8 illustrates the expression of differential genes in the gene scatter diagram in Example 1 of the present disclosure;

FIG. 9 illustrates the expression of differential genes in the gene volcano plot in Example 1 of the present disclosure;

FIG. 10 illustrates the significance analysis of gene functions in Example 1 of the present disclosure;

FIG. 11 illustrates the significance analysis of the signaling pathway in Example 1 of the present disclosure;

FIG. 12 illustrates the differential gene signaling network in Example 1 of the present disclosure;

FIG. 13 illustrates the analysis of Rattus norvegicus growth state in Example 1 of the present disclosure;

FIG. 14 illustrates the results of the prolactin content in the serum of Rattus norvegicus in each group as measured by ELISA in Example 1 of the present disclosure;

FIG. 15 illustrates the results of the IL-β content in the serum of Rattus norvegicus in each group as measured by ELISA in Example 1 of the present disclosure;

FIG. 16 illustrates the results of the pituitary protein content (n=4) of Rattus norvegicus in each group as measured by Western blot in Example 1 of the present disclosure;

FIG. 17 illustrates the action mechanism of hordenine on prolactinoma and adrenocorticotrophic hormone adenoma in Example 1 of the present disclosure;

FIG. 18 illustrates the changes in body weight (n=10) of Rattus norvegicus in Example 1 of the present disclosure;

FIG. 19 illustrates the pairwise comparison of data of Rattus norvegicus renal function index BUN and creatinine detection (n=10) of the low-dose group, the control group, and the high-dose group, and the differences all are not statistically significant (P>0.05).

FIG. 20 is pathological section staining diagrams of organs in Example 1 of the present disclosure (n=3).

### DESCRIPTION OF THE EMBODIMENTS

In order to make the objectives, technical solutions, and advantages of the examples of the present disclosure clearer, the technical solutions in the examples of the present disclosure will be described clearly and completely hereinbelow. If the specific conditions are not indicated in the examples, they shall be carried out in accordance with conventional conditions or conditions recommended by the manufacturers. If the manufacturers of the used reagents or instruments are not indicated, they all are conventional products that are commercially available.

Unless otherwise defined herein, the scientific and technical terms used in conjunction with the present disclosure shall have the meanings commonly understood by those of ordinary skill in the art. Exemplary methods and materials are described hereinbelow, but methods and materials similar or equivalent to those described herein can also be used in the practice or test of the present disclosure.

It is an object of one or more embodiments of the present disclosure to provide the use of hordenine in the preparation of an inhibitor for inhibiting a MAPK signaling pathway. The inventor has found that hordenine is pharmaceutically effective in the treatment of pituitary prolactinoma and adrenocorticotrophic hormone adenoma by inhibiting a TLR4/NF-κB/MAPK signaling pathway, solving the dilemma of medication shortage for patients of prolactinoma and adrenocorticotrophic hormone adenoma.

It is another object of one or more embodiments of the present disclosure to provide a MAPK signaling pathway inhibitor, in which a new substance has been found to inhibit a MAPK signaling pathway, and the substance has a significant inhibitory effect on the MAPK signaling pathway.

It is still another object of one or more embodiments of the present disclosure to provide the use of a MAPK signaling pathway inhibitor in the manufacture of a medicament for the treatment of pituitary tumors, and this use provides a new substance of medicament for the treatment of prolactinoma and adrenocorticotrophic hormone adenoma; the inventor has found that by inhibiting a MAPK signaling pathway, it is helpful for the treatment of prolactinoma and adrenocorticotrophic hormone adenoma.

The present disclosure is carried out as follows:

One or more embodiments of the present disclosure provide the use of hordenine or a pharmaceutically acceptable salt thereof in the preparation of an inhibitor for inhibiting a MAPK signaling pathway.

One or more embodiments of the present disclosure provide a method for inhibiting a MAPK signaling pathway, comprising contacting hordenine or a pharmaceutically acceptable salt thereof with the MAPK signaling pathway in vivo or in vitro.

One or more embodiments of the present disclosure provide hordenine or a pharmaceutically acceptable salt thereof for use as an inhibitor for inhibiting a MAPK signaling pathway.

Hordenine has a chemical name of 4-(2-dimethylaminoethyl)phenol, and a chemical structure formula of

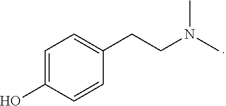

Hordenine is derived from malt. Generally, hordenine can act on adrenergic receptors, has the effects of relaxing bronchial smooth muscle, contracting blood vessels, increasing vascular pressure, raising blood pressure and stimulating central nervous system, is useful for alleviating bronchitis and bronchial asthma, and enhancing uterine tension and movement, etc., and meanwhile, protects against radiation-induced injury.

In one or more embodiments of the present disclosure, the inventor has found through inventive work that hordenine has the function of inhibiting aTLR4/NF-κB/MAPK signaling pathway. This application provides new research ideas for fields related to MAPK signaling pathways.

Specifically, MAPK, an abbreviation of mitogen-activated protein kinase, is an important transmitter for transmitting signals from cell surface into cell nucleus.

One or more embodiments of the present disclosure also provide a MAPK signaling pathway inhibitor, wherein the inhibitor comprises hordenine.

One or more embodiments of the present disclosure also provide a MAPK signaling pathway inhibitor, wherein the inhibitor comprises hordenine or a pharmaceutically acceptable salt thereof.

In addition, one or more embodiments of the present disclosure also provide the use of a MAPK signal pathway inhibitor in the manufacture of a medicament for the treatment of pituitary tumors, comprising prolactinoma and adrenocorticotrophic hormone adenoma.

One or more embodiments of the present disclosure also provide a method for treating pituitary tumors, comprising administering a MAPK signaling pathway inhibitor to a subject in need.

One or more embodiments of the present disclosure also provide the use of a MAPK signaling pathway inhibitor in the treatment of pituitary tumors.

In one or more embodiments, the pituitary tumors include prolactinoma and adrenocorticotrophic hormone adenoma.

In one or more embodiments, the MAPK signaling pathway inhibitor is a MAPK signaling pathway inhibitor as described herein.

The present disclosure provides the use of a MAPK signal pathway inhibitor as described herein in the manufacture of a medicament for the treatment of a disease associated with MAPK signaling pathway abnormality.

In one or more embodiments, the MAPK signaling pathway abnormality is TLR4/NF-κB/MAPK signaling pathway abnormality.

In one or more embodiments, the MAPK signaling pathway abnormality comprises the overexpression of at least one of MAPK12, TLR4, PRL, ACTH, IL-β, and TNF-α.

The present disclosure provides a method for treating a disease associated with MAPK signaling pathway abnormality, comprising administering a MAPK signaling pathway inhibitor as described in the present disclosure to a subject in need.

The present disclosure provides the use of a MAPK signaling pathway inhibitor as described herein in the treatment of a disease associated with MAPK signaling pathway abnormality.

In one or more embodiments, the MAPK signaling pathway abnormality is TLR4/NF-κB/MAPK signaling pathway abnormality.

In one or more embodiments, the MAPK signaling pathway abnormality comprises the overexpression of at least one of MAPK12, TLR4, PRL, ACTH, IL-β, and TNF-α.

The inventor has found through inventive work that MAPK signaling pathways, especially TLR4/NF-κB/MAPK-mediated signaling pathways, are involved in the occurrence of prolactinoma and adrenocorticotrophic hormone adenoma, and are new target for the treatment of prolactinoma and adrenocorticotrophic hormone adenoma.

The Present Disclosure Includes at Least the Following Beneficial Effects:

One or more embodiments of the present disclosure provide the use of hordenine in the preparation of an inhibitor for inhibiting a MAPK signaling pathway. It has been found that hordenine can play a pharmacological effect in the treatment of prolactinoma and adrenocorticotrophic hormone adenoma by inhibiting a TLR4/NF-κB/MAPK signaling pathway, solving the dilemma of medication shortage for patients of prolactinoma and adrenocorticotrophic hormone adenoma.

One or more embodiments of the present disclosure also provide a MAPK signaling pathway inhibitor, in which a new substance has been found to inhibit the MAPK signaling pathway, and the substance has a significant inhibitory effect on the MAPK signaling pathway.

In addition, one or more embodiments of the present disclosure also provide the use of a MAPK signaling pathway inhibitor in the manufacture of a medicament for the treatment of prolactinoma and adrenocorticotrophic hormone adenoma, and this use provides a new substance of medicament for the treatment of prolactinoma and adrenocorticotrophic hormone adenoma, which is helpful for the treatment of prolactinoma and adrenocorticotrophic hormone adenoma by inhibiting the MAPK signaling pathway.

The use of hordenine in the preparation of an inhibitor for inhibiting a MAPK signaling pathway in one or more embodiments of the present disclosure is specifically described hereinbelow.

One or more embodiments of the present disclosure provide the use of hordenine in the preparation of an inhibitor for inhibiting a MAPK signaling pathway.

In one or more embodiments, the above MAPK signaling pathway is TLR4/NF-κB/MAPK signaling pathway.

In one or more embodiments, the above inhibition of MAPK signaling pathway is used for inhibiting the expression of PRL. Specifically, it means that hordenine or a MAPK signaling pathway inhibitor can restore the super-high expression level of PRL in pituitary patients to or close to the normal level.

In one or more embodiments, the above inhibition of MAPK signaling pathway is used for inhibiting the expression of ACTH. Specifically, it means that hordenine or a MAPK signaling pathway inhibitor can restore the super-high expression level of ACTH in pituitary patients to or close to the normal level.

In one or more embodiments, the above inhibition of MAPK signaling pathway is used for inhibiting the expression of MAPK12. Specifically, it means that hordenine or a MAPK signaling pathway inhibitor can restore the super-high expression level of MAPK12 in pituitary patients to or close to the normal level.

In one or more embodiments, the above inhibition of MAPK signaling pathway is used for inhibiting the expression of TLR4. Specifically, it means that hordenine or a MAPK signaling pathway inhibitor can restore the super-high expression level of TLR4 in pituitary patients to or close to the normal level.

In one or more embodiments, the above inhibition of MAPK signaling pathway is used for inhibiting the expression of TNF-α. Specifically, it means that hordenine or a MAPK signaling pathway inhibitor can restore the super-high expression level of TNF-α in pituitary patients to or close to the normal level.

In one or more embodiments, the above inhibition of MAPK signaling pathway is used for inhibiting the expression of IL-β. Specifically, it means that hordenine or a MAPK signaling pathway inhibitor can restore the super-high expression level of IL-β in pituitary patients to or close to the normal level.

One or more embodiments of the present disclosure also provide a MAPK signaling pathway inhibitor, wherein the inhibitor comprises hordenine.

In one or more embodiments, the MAPK signaling pathway is a TLR4/NF-κB/MAPK-mediated signaling pathway.

In one or more embodiments, a MAPK signaling pathway inhibitor is used for inhibiting the expression of at least one of MAPK12, TLR4, PRL, ACTH, IL-β, and TNF-α.

One or more embodiments of the present disclosure also provide the use of a MAPK signal pathway inhibitor in the manufacture of a medicament for the treatment of prolactinoma and adrenocorticotrophic hormone adenoma.

In one or more embodiments, the MAPK signaling pathway inhibitor is the above MAPK signaling pathway inhibitor.

In addition, one or more embodiments of the present disclosure also provide the use of the above MAPK signal pathway inhibitor in the manufacture of a medicament for the treatment of a disease associated with MAPK signaling pathway abnormality.

In one or more embodiments, the MAPK signaling pathway abnormality is TLR4/NF-κB/MAPK signaling pathway abnormality.

In one or more embodiments, the MAPK signaling pathway abnormality comprises the overexpression of at least one of MAPK12, TLR4, PRL, ACTH, TNF-α, and IL-1β.

Pituitary prolactinoma and adrenocorticotrophic hormone adenoma occur in the anterior pituitary gland, which integrates hormone signaling pathways that control the reproductive and growth functions of thyroid, adrenal glands, and the like. The pituitary gland accurately regulates the homeostasis of the internal environment by promoting hormone secretion, but the resulting pituitary signal may also cause a shaped pituitary growth response, including functional pituitary cell dysplasia, hyperplasia and adenoma formation. At present, the specific pathogenesis of this common tumor is still not fully understood. The inventor of the present disclosure has found through inventive work and research that the occurrence of prolactinoma and adrenocorticotrophic hormone adenoma is related to the regulation of signal protein pathways, which specifically are MAPK signaling pathways, and more typically, TLR4/NF-κB/MAPK signaling pathways.

In an inhibitor, the dose of hordenine is 10 to 40 mg, e.g., 11 to 39 mg, 12 to 38 mg, 13 to 37 mg, 14 to 36 mg, 15 to 35 mg, 16 to 34 mg, 17 to 33 mg, 18 to 32 mg, 19 to 31 mg, 20 to 30 mg, 21 to 29 mg, 22 to 28 mg, 23 to 27 mg, 24 to 26 mg, or 25 mg.

One or more embodiments of the present disclosure also provide a pharmaceutical composition, comprising hordenine or a pharmaceutically acceptable salt thereof, and a pharmaceutically acceptable carrier.

In one or more embodiments, the pharmaceutical composition further comprises an additional active agent for treating pituitary tumors, such as pituitary prolactinoma and/or adrenocorticotrophic hormone adenoma. In one or more embodiments, the additional active agent includes bromocriptine and/or bromocriptine mesylate.

In one or more embodiments, a pharmaceutical composition comprising hordenine or a pharmaceutically acceptable salt thereof is formulated for oral delivery, topical delivery, or parenteral delivery.

In one or more embodiments, the subject described herein is an animal, for example, a mammal, such as a human.

The term “pharmaceutically acceptable salt” as used herein means a salt or a zwitterionic form of hordenine of the present disclosure, which is suitable for the treatment of diseases without excessive toxicity, irritation and allergic reactions; it is compatible with a reasonable benefit-to-risk ratio and effective for its intended use. In one or more embodiments, pharmaceutically acceptable salts of hordenine include acid addition salts of hordenine, e.g., hydrochlorides, acetates, adipates, alginates, citrates, aspartates, benzoates, benzenesulfonates, bisulfates, butyrates, camphorates, camphorsulfonates, bisgluconates, glycerophosphates, hemi sulfates, heptylates, caproates, formates, fumarates, hydrochlorides, hydrobromides, hydroiodides, 2-hydroxyethanesulfonates (hydroxyethyl sulfonates), lactates, maleates, mesitylene sulfonates, methanesulfonates, naphthalen esulfonates, nicotinates, 2-naphthalenesulfonates, oxalates, pamoates, pectinates, persulfates, 3-phenylpropionic acid, picrates, pivalates, propionates, succinates, tartrates, trichloroacetates, trifluoroacetates, phosphates, glutamates, bicarbonates, p-toluenesulfonates and/or undecanoates.

As used herein, “pharmaceutically acceptable carrier” refers to any and all physiologically compatible solvents, dispersion media, coatings, antibacterial and antifungal agents, isotonic and absorption delaying agents, and the like. Examples of pharmaceutically acceptable carriers include one or more of water, saline, phosphate buffered saline, dextrose, glycerol, ethanol, and the like, and a combination thereof.

As used herein, “treatment” refers to, for example, the stagnation of symptoms, prolongation of survival time, improvement of partial or all symptoms, and eradication of partial or all conditions, diseases, or disorders in a subject (e.g., a mammal). In some cases, the therapeutic effect may also be preventive.

In one or more embodiments, the pituitary adenoma is at least one selected from the group consisting of prolactinoma (PRL), adrenocorticotrophic hormone adenoma (ACTH), growth hormone adenoma (GH), thyroid stimulating hormone adenoma (TSH), pituitary gonadotropin adenoma (PGA) and mixed hormone secreting adenoma.

The features and properties of the present disclosure are further described in detail hereinbelow in conjunction with examples.

### Example 1

I. Establishment of Tumor Model

1.1. Establishment of Rattus norvegicus Prolactinoma Model.

The Rattus norvegicus strains used in the Rattus norvegicus pituitary prolactinoma model mainly include Fischer344 (F344) [34] rats, wistar-furth(Wister) rats, SD rats, and the like. The inventor has found through experimental research that compared with Wister rats and SD rats, F344 rats are more sensitive, and have a shorter tumor formation time for the induced prolactinoma model; the tumor formation rate is up to 100%, and the operation is simple and easy; moreover, there is no tumor metastasis or generation of associated tumors; it is an ideal animal model for studying pituitary prolactinoma.

Eighty F344 rats are randomly divided into a normal group and a model group. All rats in the other group except those in the normal group are used as prolactinoma models.

The model group needs to be ovariectomized first: The Rattus norvegicus is anesthetized, and placed on a fixed table on its belly; the long hair around the back of the ribs is cut off, and the skin is wiped with iodine for disinfection; a longitudinal incision of about 1 to 2 cm is made from the lumbar spine down the midline of the back; the skin is cut open; round-tip scissors are inserted between the skin and the muscle, which expand and peel off the muscle from the skin; the psoas muscles are cut open about 1 cm away from the spine and along the scapular line under the left and right ribs, respectively, to form an incision of about 1 cm in length, which immediately reveals the fat tissue surrounding the ovaries on both sides; the fat tissue is grasped with tweezers and gently pulled out of the incision to expose the pale pink ovaries and the closely connected uterine horns; the uterine horns are cut off with scissors and the ovaries are removed; bleeding is checked; the fat tissue is pushed back into the abdominal cavity; the wound is glued with medical tape and then an amount of penicillin is injected; the Rattus norvegicus is put back into the cage; food and water are provided normally one week after the operation.

One week after removing the ovaries on both sides, rats in the model group are intraperitoneally injected with an estradiol benzoate injection (0.1 mg/kg) every 5 days; rats in the normal group do not do any treatment, eat and drink freely, and are given 12 hours of light every day.

1.2. Observation of Survival Status of Model Animals.

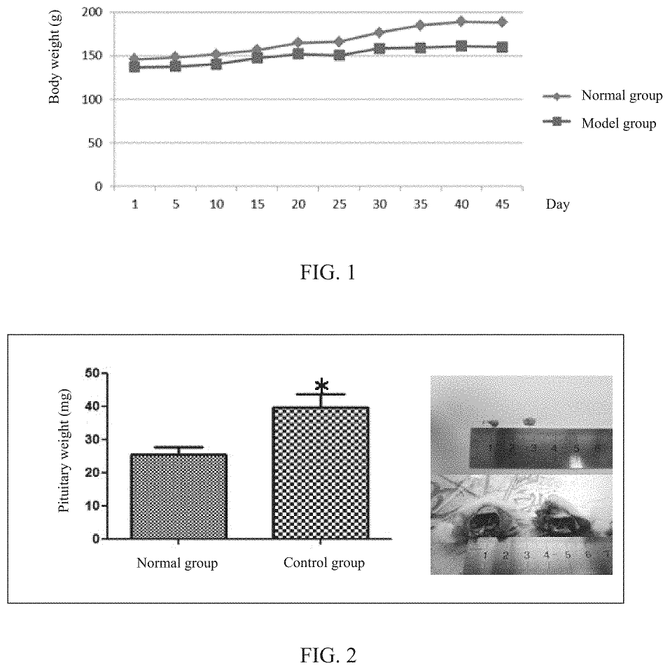

The eating and activity conditions of the model animals are observed every day; the weight of rats is weighed and recorded every five days. The average weight of rats is normally increased as shown in FIG. 1. Within 45 days of medication, some of the rats in the model group show signs of loss of appetite, lethargy, weight loss, lack of movement, and the like; the rats in the normal group show no abnormalities.

1.3. Collection of Pituitary Tumor Specimens.

The animals in the normal group and the model group are anesthetized separately to remove the pituitary glands 45 days after estrogen injection, which are kept at −80° C. for later use. Four F344 rats are taken from the normal group and the model group, respectively. The average weight of the pituitary glands in the normal group is 25.5 mg; the average weight of the pituitary glands in the model group is 39.75 mg, which is about 1.5 times that of the pituitary glands in the normal group, as shown in FIG. 2. As shown by magnetic resonance imaging (MRI) and its data, after stimulation with estradiol, the volume of the pituitary glands of the model group is increased significantly, and is about 3 times the volume of the pituitary glands of the normal group.

1.4. Western Blotting Analysis Results.

The PRL protein expression in the pituitary glands of the normal and model animals is detected by Western Blotting method.

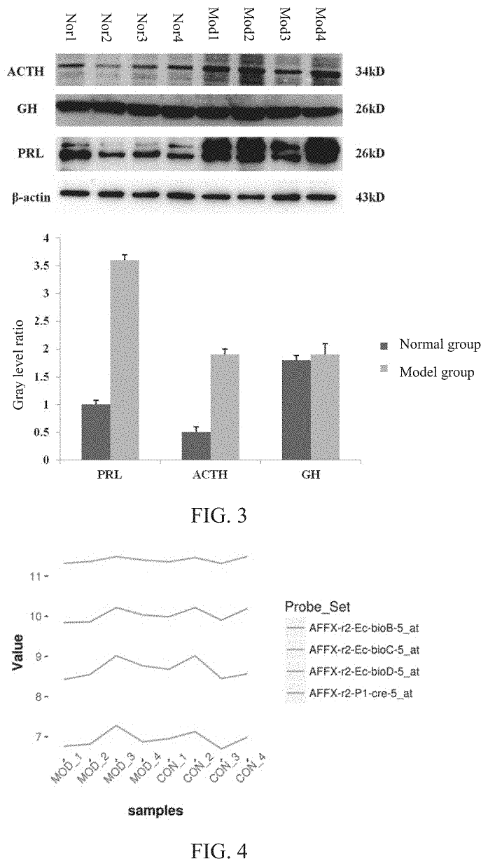

The statistical methods use SPSS22.0 software and GraphPad Prism 5.01 for statistical analysis and graphing; the measurement data are represented by x̅±s; the comparison of sample means between the two groups is performed by T test; P<0.05 indicates that the difference is statistically significant. Compared with the rats of the normal group, the expression of PRL and ACTH proteins in the pituitary glands of the rats of the model group increases significantly, as shown in FIG. 3.

II. Analysis of Pituitary Tumor-Associated Pathways and Genes

Gene chips are used to screen signaling pathways and relevant genes thereof that are highly associated with the occurrence of pituitary tumors.

2.1 Four pituitary tumor model rats established in Step 1.1 “Establishment of Rattus norvegicus pituitary tumor model” and four normal rats of the control group are used as samples. This step is entrusted to be completed by Beijing Cnkingbio Biotechnology Co. Ltd.

Quality Control of Sample RNA

The RNA concentration meets the requirements of the Clariom S chip experiment; the initial RNA concentration is at least >17 ng/μL; the total amount is at least >50 ng; 260/280 is generally in the range of 1.8 to 2.1; the sample is free of macromolecular contamination; and the sample remains intact without degradation, as shown in Table 1.

TABLE 1
RNA quality control

| | | | | | | | Presence |
|---|---|---|---|---|---|---|---|
| Sam- | | Concen- | | | | Quality | of |
| ple | Sample | tration | | Volume | Total | control | residual |
| No. | Name | (ng/μL) | 260/280 | (μL) | (μg) | result | sample |
| 1 | Nor 1 | 2826 | 1.98 | 18 | 50.87 | A | No |
| 2 | Nor 2 | 1963 | 1.97 | 18 | 35.33 | A | No |
| 3 | Nor 3 | 807 | 1.98 | 18 | 14.53 | A | No |
| 4 | Nor 4 | 2276 | 1.92 | 18 | 40.97 | A | No |
| 5 | Mod 1 | 5065 | 1.97 | 18 | 91.17 | A | No |
| 6 | Mod 2 | 3363 | 1.94 | 18 | 60.53 | A | No |
| 7 | Mod 3 | 6923 | 2.01 | 18 | 124.61 | A | No |
| 8 | Mod 4 | 5787 | 2.00 | 18 | 104.17 | A | No |

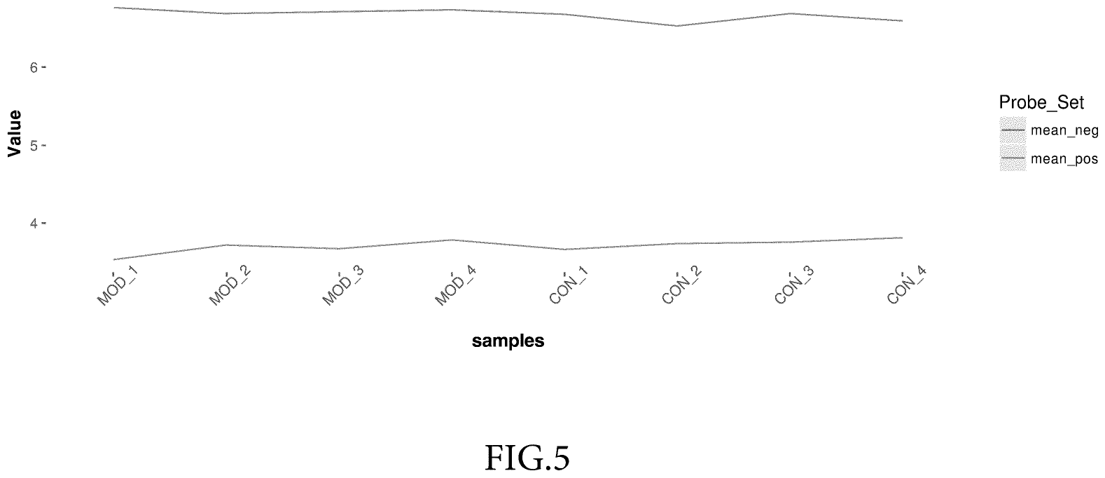

The chip quality control results are good, as shown in FIG. 4 and FIG. 5.

Difference Analysis

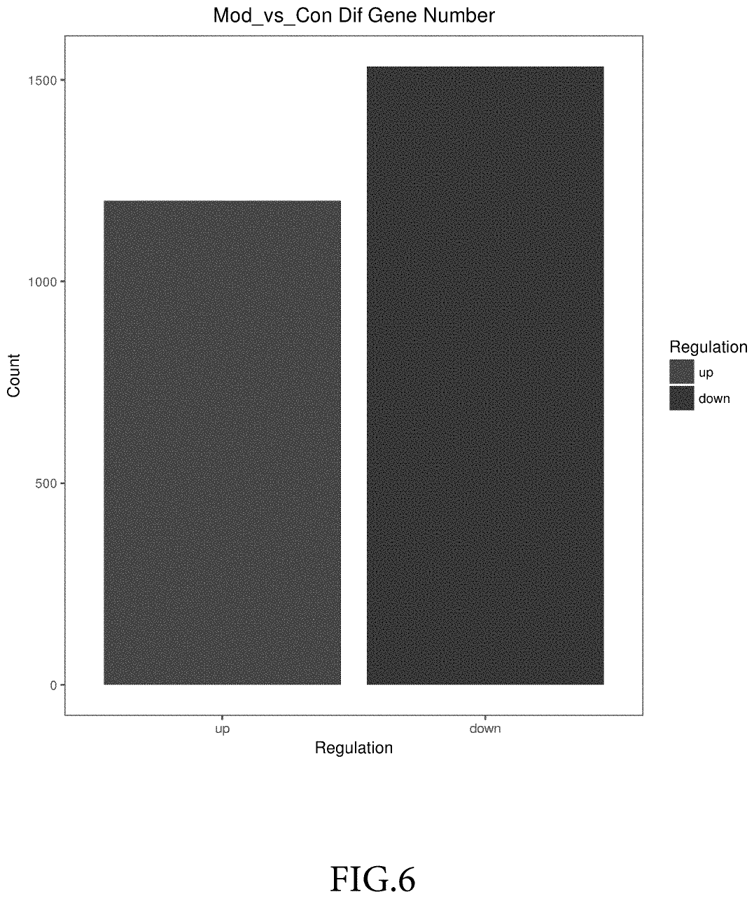

Compared with the control group, the number of up-regulated genes is 1199, and the number of down-regulated genes is 1534 in the model group, as shown in FIG. 6.

2.2. Western Blotting Verification of Highly Expressed Genes Screened by Gene Chip

Compared with the normal group, the expression of MAPK12 in the pituitary tissues of rats in the pituitary tumor model group is significantly increased (**P<0.01).

2.3. Application of Statistical Methods

The statistical methods use SPSS22.0 software and GraphPad Prism 5.01 for statistical analysis and graphing; the measurement data are represented by x̅±s; the comparison of sample means between the two groups is performed by T test; P<0.05 indicates that the difference is statistically significant.

2.3.1. Difference Diagram Analysis

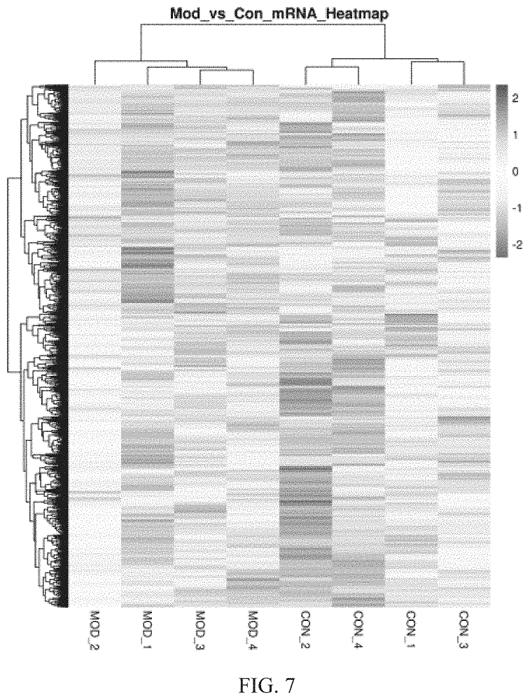

Clustering diagram: In order to completely and intuitively show the relationships and differences between samples, the differentially expressed genes are hierarchically clustered and displayed in the form of heat maps. The correlation between samples is calculated by the expression of selected differential genes, and samples of the same type can be gathered in the same cluster. Genes clustered in the same cluster may have similar biological functions. Reference can be made to FIG. 7.

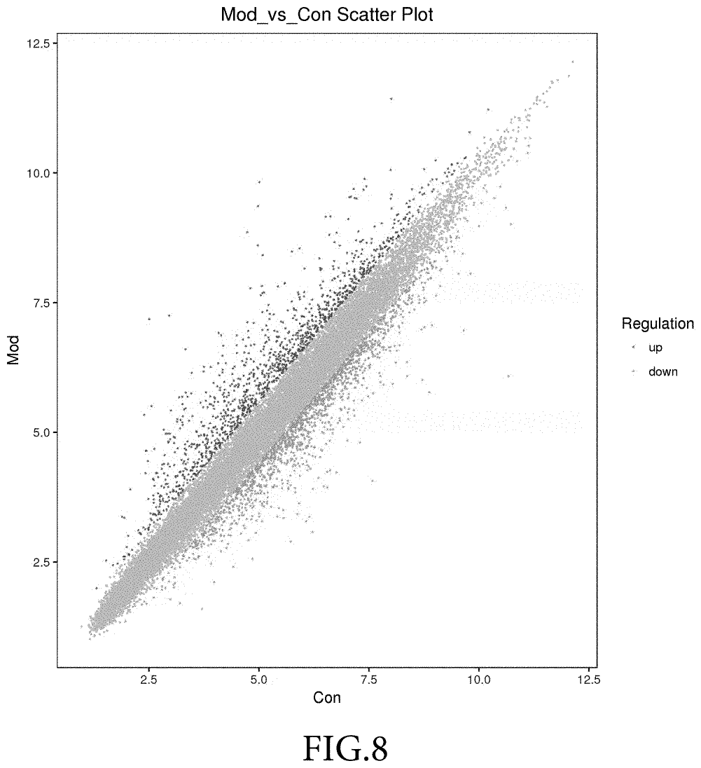

Scatter diagram: The horizontal and vertical coordinates respectively represent the log 2 value of the expression amount of two samples, showing the profile of gene up-regulation and down-regulation, as shown in FIG. 8.

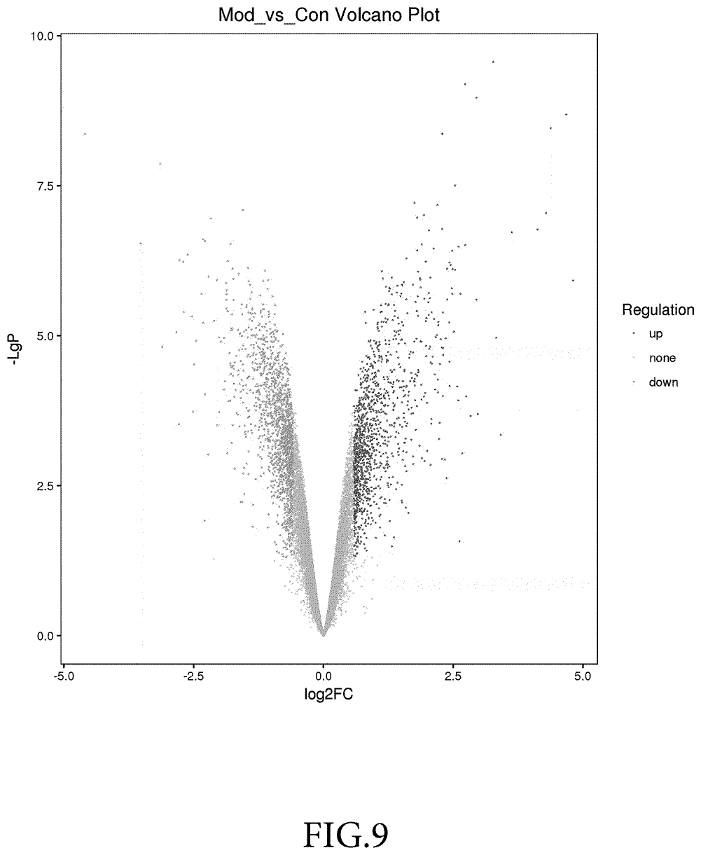

Volcano plot: The chip or sequencing data is analyzed by T-Test to obtain P value and FC value. The two factors jointly create a Volcano Plot to show the significant difference between the data of the two samples. The horizontal axis represents the fold of difference of the probe (Fold chang), and the vertical axis represents the significance of difference of the probe (−log 10 P-value), as shown in FIG. 9.

2.3.2. In-Depth Analysis of Differential Genes

Significance Analysis of Function-GO Enrichment Analysis

Gene Ontology database is called GO database for short, which is a cross-species, comprehensive and descriptive database. Its purpose is to establish a semantic vocabulary standard that is suitable for various species, defines and describes the functions of genes and proteins, and can be updated with the continual development of research. The vocabulary of genes and gene products involved in gene ontology is divided into three categories, covering three aspects of biology: cellular component; molecular function; and biological process. The GO database clarifies the hierarchical relationship between gene functions and can help us better understand the relationship between gene functions. The method of screening out the significant, accurate and targeted gene function representing the target gene group based on the GO database is called GO Enrichment Analysis. Its value lies in discovering the most important function of the trait carried by the target gene, discovering the main or non-main function of the same gene in this trait, and judging whether the research target is accurate under a larger number of samples. The significance level (P-value) and false determination rate (FDR) of each function are calculated by Fisher's exact test and multiple comparison test. In this way, the significant functions demonstrated by the gene are screened out, and the standard of significance screening is: P value <0.01.

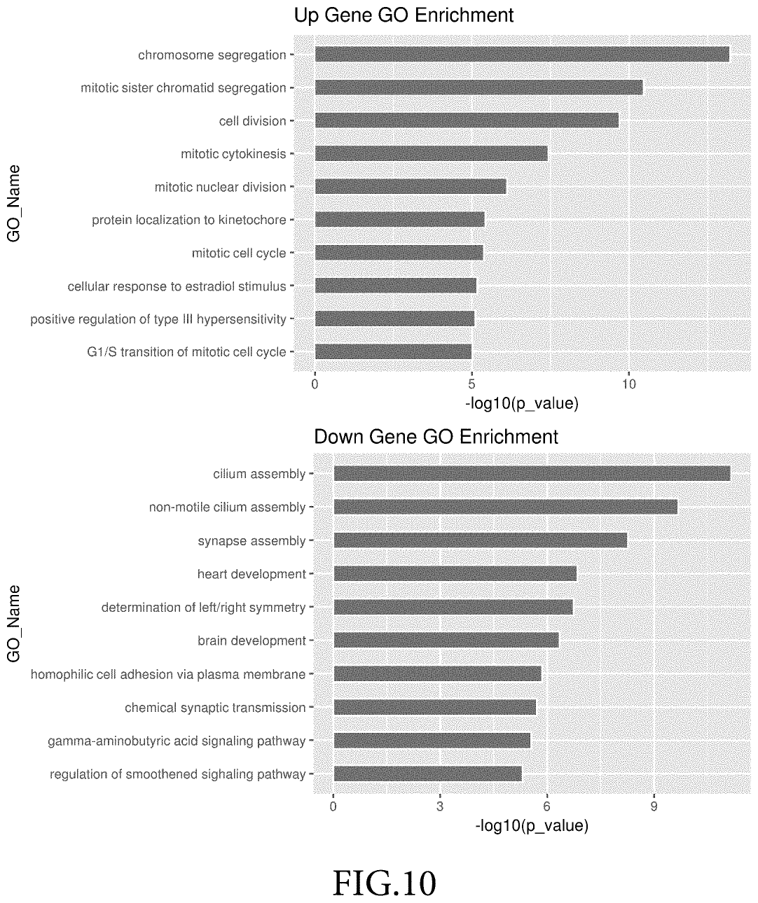

By chip screening and GO database analysis, it is shown that the top three relationships between the highly expressed gene functions in the model group are chromosome segregation, sister chromatid segregation, and cell division compared with the control group. All involve cell division and other relevant functions. Reference can be made to FIG. 10.

Significance Analysis of Signal Pathway-Pathway Enrichment Analysis

Kyoto Encyclopedia of Genes and Genomes (KEGG) is a database that systematically analyzes the relationship between genes (and coded products thereof), gene functions, and genomic information, and helps researchers study genes and expression information as a whole network. Genomic information is stored in the Genes database, and more advanced functional information is stored in the Pathway database, including graphical cell biochemical processes such as metabolism, membrane transport, signal transmission, cell cycle, and conserved sub-pathways, etc. The integrated metabolic pathway query provided by KEGG is excellent, including the metabolism of carbohydrates, nucleosides, amino acids, and the like, as well as the biodegradation of organic matters, which not only provides all possible metabolic pathways, but also comprehensively analyzes enzymes that catalyze each step of the reaction. KEGG is a powerful tool for metabolic analysis and metabolic network research in organisms. The obtained differential genes are subjected to signaling pathway annotation by Pathway Enrichment Analysis method based on the KEGG database to obtain all signaling pathways in which the gene participates. Subsequently, the significance level (P-value) and false determination rate (FDR) of each signaling pathway are calculated by Fisher's exact test and multiple comparison test. In this way, the significant signaling pathways demonstrated by the gene are screened out, and the standard of significance screening is: P value <0.05.

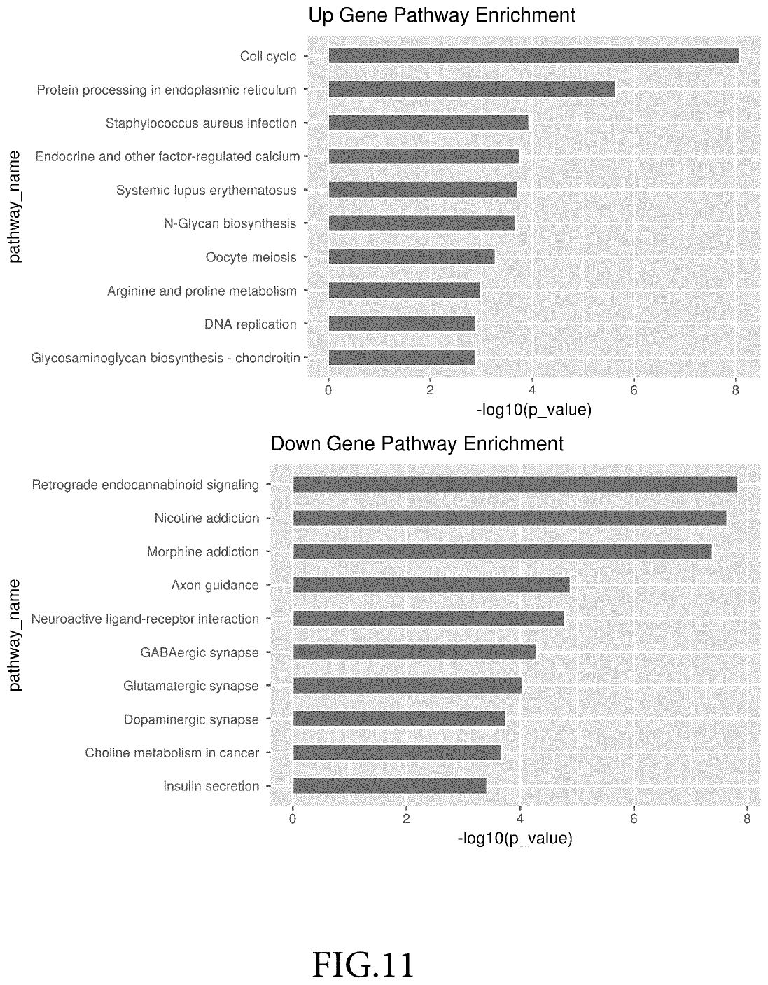

By gene chip screening and KEGG database analysis, it is shown that the top three significant signal pathways demonstrated by the gene are cell circulation, endoplasmic reticulum protein processing, and Staphylococcus aureus infection compared with the normal group, as shown in FIG. 11.

2.3.3. Determination of Differential Genes

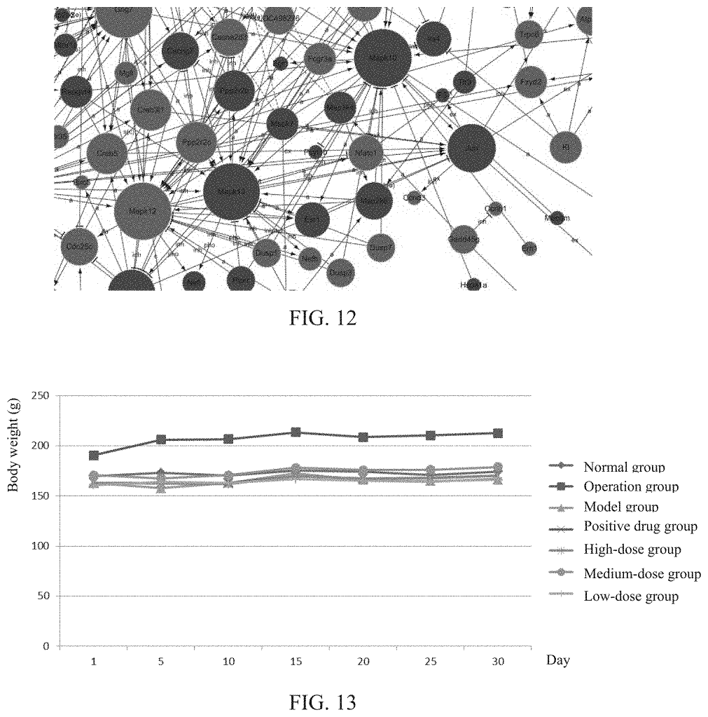

By gene chip screening in combination with the above difference analysis results, it leads to genes that are closely related to our research direction and are highly expressed, wherein MAPK12 is significantly up-regulated, as shown in FIG. 12.

Result Analysis

The biological processes leading to the onset of pituitary tumors mainly include cell division, protein processing and other biological processes that involve cell proliferation, which are indeed closely associated with the occurrence of tumors. Among the differentially expressed genes in the normal group and the model group, the high expression of MAPK12 and its differential expression from other genes have been found. The inventor infers that TLR4 receptors on microglia in the pituitary glands may be activated by certain stimuli, which therefore activates TLR4/NF-Kb/MAPK12 signaling pathway and causes tumor cell proliferation. Microglias are macrophages of the central nervous system. The abnormal activation of glial cells and the neuroinflammatory response caused by the release of various pro-inflammatory factors are often detrimental to brain health.

III. Drug Efficacy of Hordenine on Pituitary Prolactinoma and Adrenocorticotrophic Hormone Adenoma

3.1. Drug Efficacy of Hordenine on Rattus Norvegicust Pituitary Tumors

70 rats of the prolactinoma and adrenocorticotrophic hormone adenoma model as established in Step 1.1 “Establishment of Rattus norvegicus pituitary tumor model” and normal rats of the control group are used as samples.

The 70 F344 rats are randomly divided into the normal group, operation group, model group, positive drug group (bromocriptine positive drug group), high-dose hordenine group, medium-dose hordenine group, and low-dose hordenine group, 8 rats in each group. Except rats in the normal group and the operation group, rats in the other groups are all made into rats with prolactinoma and adrenocorticotrophic hormone adenoma.

Rats in the positive drug group are given an aqueous bromocriptine solution by gavage at a dose of 0.45 mg/kg. Rats in the high-, medium- and low-dose groups of an aqueous solution of hordenine are given an aqueous solution of hordenine by oral gavage at a dose of 40 mg/kg, 20 mg/kg, and 10 mg/kg, respectively. Rats in the normal group and the model group are given the same dose of distilled water. The administration is performed once a day for 30 days, and during this period of time, the rats are weighed every five days and the changes in body weight are recorded.

Specimens Collection from the Above 70 Samples:

Serum collection: 24 hours after the last administration, the rats are fasted for 12 hours, but free to drink. In the morning of the next day, blood is taken from the eyeballs of rats in each group, 2 ml for each, which is subjected to centrifugation to collect serum for testing.

Tissue extraction: The animals in each group are anesthetized, and the pituitary glands are removed as described in Section 2.2.2 of Part I, which are kept at −80° C. for later use.

The rats in each group all grow normally as shown in FIG. 13. The rats of the operation group rapidly gain weight. Some of the rats in the model group and the treatment groups show loss of appetite, lethargy, weight loss, and lack of movement. The rats in the normal group and the operation group show no abnormalities.

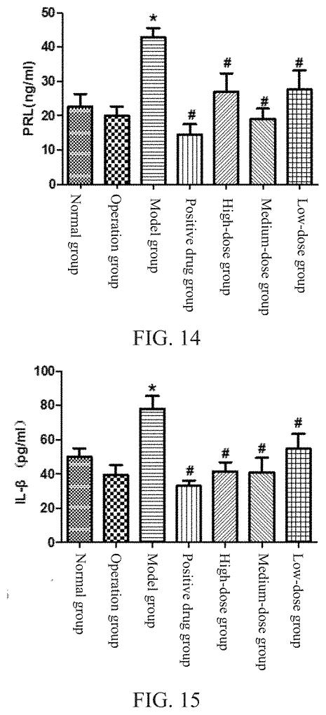

The contents of PRL and IL-β in the serum of rats in each group are determined by ELISA according to the procedures in the ELISA kit instructions. The contents of prolactin (PRL) and IL-β in the serum of rats in each group as determined by ELISA are shown in FIG. 14 and FIG. 15. It can be seen from FIG. 14 and FIG. 15 that hordenine can significantly inhibit the expression of PRL and IL-β in the model group, and restore PRL and IL-β to or close to normal levels.

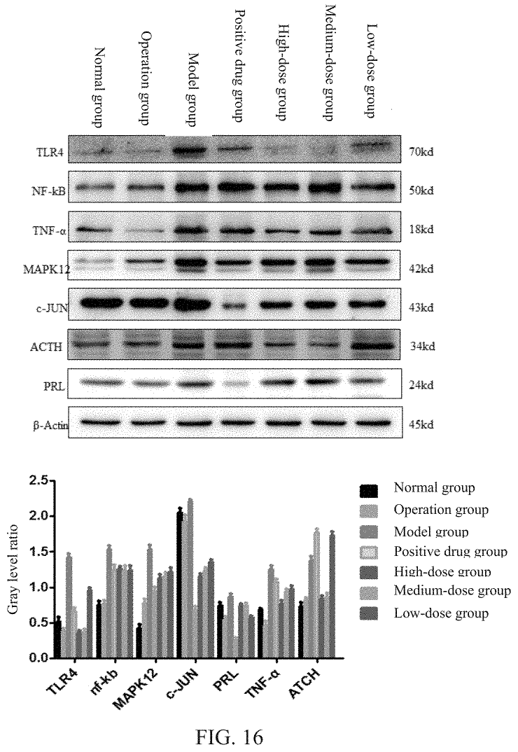

The protein expression of TLR4/NF-κB/MAPK pathway in rats in the treatment groups after the administration of hordenine is determined by Western blotting detection. The effect of hordenine on the expression of various proteins in the pituitary glands of rats with prolactinoma and adrenocorticotrophic hormone adenoma as determined by Western blot detection on is shown in FIG. 16. It can be seen from FIG. 16 that hordenine can significantly reduce the expression of TLR4, MAPK12, PRL, TNF-α and ATCH in the model group, and restore it to normal levels.

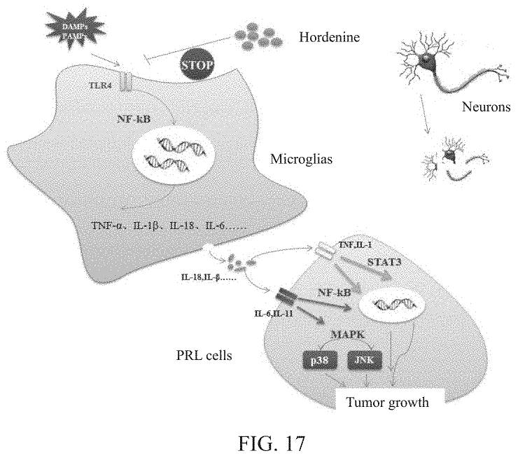

The above results show the effect of hordenine on Rattus norvegicus prolactinoma and adrenocorticotrophic hormone adenoma. The action mechanism of hordenine on prolactinoma and adrenocorticotrophic hormone adenoma is shown in FIG. 17.

NF-κB signaling pathway is a classic pathway for tumorigenesis and development. The NF-κB pathway can be activated by various signals including components of multiple pathogens such as lipopolysaccharides, proinflammatory cytokines such as TNF, IL-1, and mitogen, etc., so that the NF-κB is translocated into the nucleus and bind to relevant DNA motifs to induce the transcription of target genes. After the administration of hordenine and bromocriptine, the expression of TLR4, NF-kB, caspase-1, MAPK12, and STAT3 proteins in the brain pituitary glands of rats with prolactinoma and adrenocorticotrophic hormone adenoma is significantly down-regulated compared with the model group. Therefore, the inventor believes that hordenine inhibits the expression of TLR4 protein in this pathway, thereby inhibiting tumor growth.

IV. Acute Toxicity Test of Hordenine

Experimental Animals

The experimental animals are 40 SPF-grade SD rats, provided by the Experimental Animal Center of China Three Gorges University (SCXK (E)) 2017-0012), wherein half are males, and half are females, each with a body weight of 150 g; the animal number is: No. 42010200001245, and the experimental unit license number is: SYXK (E) 2014-0080.

5 SPF-grade SD rats are randomly selected for toxicity prediction. Another 30 SPF-grade SD rats are randomly selected, wherein half are males, and half are females. After the purchase of the animals, the animals are adaptively fed for one week. The rats are fasted at the night before the experiment (without stopping the provision of water). During the experiment, the animals are randomly divided into 3 groups, which are the control group, the low-dose hordenine treatment group (0.035 g/kg), and the high-dose hordenine treatment group (0.07 g/kg).

Toxicity Prediction

7 mg/ml of hordenine solution (the theoretical maximum solubility of hordenine is 7 mg/ml) is used for pre-experiment to observe the survival of rats. 5 SPF-grade SD rats are randomly selected, and hordenine is administered by gavage at the maximum dose of 0.07 g/kg, twice a day, with an interval of no less than 4 hours, 1 day of administration, and 7 days of observation. The poisoning, death, and amounts of food and water consumed by the rats are daily observed and recorded. For rats that die during the experiment, they are immediately dissected to find the cause of death. After the completion of the experiment, all dying rats will be dissected to remove the heart, liver, spleen, lung and kidney for visual observation, and pathological sections of each organ are made to roughly determine the target organ of toxicity.

Results show that no rats die within 7 days; general clinical signs of rats include: After the treatment, the activity of rats is relatively reduced and recovered after about 2 hours; the hair growth is normal and shiny; the skin color is normal; and there is no abnormal secretion; the behavior, eating and drinking are all normal; no sign of flatulence; and there are no other adverse reactions.

Acute Toxicity Test

If no rats die in the pre-experiment, according to the acute toxicity test method, the maximum dose method is used, and hordenine is administered by gavage at a high dose of 0.07 g/kg and a low dose of 0.035 g/kg, respectively. The control group is given the same volume of saline. The administration is carried out twice a day (with an interval of no less than 4 hours) for 7 days, and the rats are fasted for 3 to 4 hours after the administration. The animals' immediate reactions are recorded, and the rats' appearance, behavior, activity, spirit, appetite, fur, breathing, weight, and the like, are carefully observed continuously for 14 days. The poisoning, death, and amounts of food and water consumed by the rats are recorded daily to determine the LD0 (0% mortality), LD100 (100% mortality) and the r value between the corresponding dose groups, and determine the LD50 (half lethal dose) value. For mice that die during the experiment, they should be dissected immediately to find the cause of their death. After the completion of the experiment, all survived rats are anesthetized by intraperitoneal injection of urethane, and immediately dissected to remove the heart, liver, spleen, lung, and kidney, which are observed with naked eyes for disease. And pathological sections of each organ are made: The tissues are fixed with a solution of 4% paraformaldehyde, conventionally sampled, dehydrated, embedded in paraffin, made into a slice (4 μm thick), stained with HE staining method, observed with an optical microscope, and photographed to roughly determine the target organs of toxicity. The serum of all experimental rats is used for the determination of routine blood biochemical indexes.

Results of Acute Toxicity Test:

No rats die within 7 days. Due to concentration and volume limitations, the median lethal dose (LD50) cannot be determined.

General clinical signs of rats include: After the treatment, the activity of rats is relatively reduced and recovered after about 2 hours; the hair is normal and shiny; the skin color is normal; and there is no abnormal secretion; the behavior, eating and drinking are all normal; no sign of flatulence; and there are no other adverse reactions.

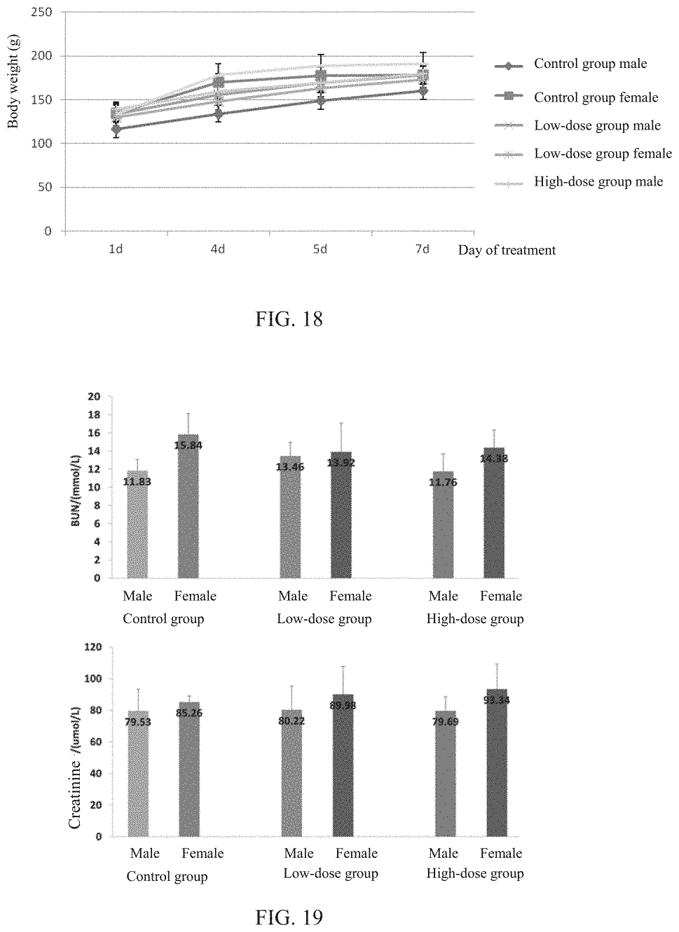

Body weight of rats: The rats are conventionally fed for one week after the treatment, and the body weight shows a physiological increase as shown in FIG. 18.

The renal function of rats is tested as shown in FIG. 19 by measuring the blood BUN and CREA levels. The differences in the renal function of rats in the three groups are statistically analyzed with spss software; there is no statistical significance in pairwise comparison between the three groups (P>0.05), indicating that the drug has no significant influence on the renal function of rats.

The liver function of rats is tested as shown in Table 2 by measuring aspartate aminotransferase (AST), alanine aminotransferase (ALT), bilirubin (BIL), alkaline phosphatase (ALP), serum albumin (ALB), total protein (TP), total bile acid (TBA), and γ-glutamyl transpeptidase (γ-GT). The differences in the liver function index of rats in the three groups are statistically analyzed with spss software; there is no statistical significance in pairwise comparison between the three groups (P>0.05), indicating that the drug has no significant influence on the liver function of rats.

TABLE 2
Rattus norvegicus serum liver function biochemical indexes

| | Control group (n = 10) | | Low-dose group (n = 10) | | High-dose group (n = 10) | |
|---|---|---|---|---|---|---|
| Group | Male | Female | Male | Female | Male | Female |
| AST/(U/L) | 142.16 ± 32.98 | 110.5 ± 20.36 | 116.65 ± 18.14 | 146.92 ± 24.61 | 147.02 ± 17.42 | 141 ± 12.04 |
| ALT/(U/L) | 52.12 ± 4.63 | 38.5 ± 7.8 | 40.46 ± 5.55 | 43.54 ± 3.84 | 51.47 ± 6.19 | 51.25 ± 4.84 |
| DBIL/(μmol/L) | 7.36 ± 2.91 | 7.34 ± 0.44 | 6.81 ± 2.83 | 9.03 ± 5.05 | 6.14 ± 0.86 | 7.23 ± 1.72 |
| TBIL/(μmol/L) | 10.52 ± 4.21 | 10.18 ± 1.06 | 10.64 ± 4.19 | 11.67 ± 5.16 | 10.08 ± 2.69 | 13.4 ± 5.73 |
| ALP/(U/L) | 268.74 ± 34.27 | 231.42 ± 31.1 | 328.05 ± 53.25 | 229.4 ± 70.39 | 278.67 ± 38.64 | 195.08 ± 92.02 |
| ALB/(g/L) | 29.09 ± 1.14 | 30.63 ± 0.84 | 28.86 ± 0.55 | 31.88 ± 1.02 | 29.5 ± 1.05 | 32.45 ± 1.61 |
| TP/(g/L) | 46.93 ± 2.03 | 52.53 ± 1.45 | 47.34 ± 1.99 | 53.54 ± 1.24 | 49.11 ± 2.17 | 55.45 ± 2.31 |
| TBA/(μmol/L) | 27.86 ± 4.59 | 24.53 ± 7.95 | 36.99 ± 10.95 | 23.77 ± 6.9 | 41.43 ± 10.19 | 27.57 ± 12.2 |
| γ-GT/(U/L) | 2.74 ± 1.13 | 2.18 ± 2.16 | 2.59 ± 1.84 | 3.92 ± 1.78 | 3.9 ± 2.10 | 3.62 ± 1.97 |

Pathological Sections of Each Organ

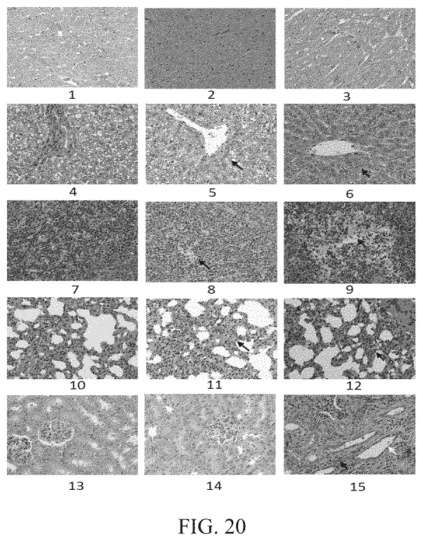

The rats are dissected, and the organs of heart, liver, spleen, lung and kidney are observed with naked eyes. The pathological sections of each organ are analyzed; the results show that the liver, spleen, lung, and kidney of the treatment groups have slight toxicity as shown in FIG. 20.

(1) Hepatocytes in the low-dose group exhibit extensive edema, cell swelling, and loose cytoplasm which is lightly stained, as shown by the black arrow 5 in FIG. 20; there are no other obvious abnormalities in the liver tissue; the Rattus norvegicus hepatocytes in the high-dose group exhibit extensive severe edema, cell swelling, loose cytoplasm which is lightly stained or vacuolated, as shown by the black arrow 6 in FIG. 20;

(2) A small amount of lymphocytes exhibit necrosis, apoptosis, nuclear pyknosis and deep staining, or fragmentation and dissolution in the white pulp in the spleen tissue of rats in the low-dose group, as shown by the black arrow 8 in FIG. 20; topical lymphocytes in the white pulp of the spleen tissue are reduced in the high-dose group, whereby a small amount of loose connective tissue is seen, as shown by the black arrow 9 (right) in FIG. 20, and a small amount of lymphocytes show necrosis, apoptosis, and nuclear fragmentation, as shown the red arrow 9 (left) in FIG. 20;

(3) The lung tissue of rats in the low-dose group exhibits a large number of proliferated alveolar epithelial cells, and the alveolar walls are thickened, as shown by the black arrow 11 in FIG. 20; no obvious abnormalities are seen in other tissues. The rats of the high-dose group are seen to have proliferated alveolar epithelial cells, and the alveolar walls are thickened, as shown by the black arrow 12 (bottom) in FIG. 20; a very small amount of infiltrated granulocytes can be seen around the blood vessels, as shown by the red arrow 12 (top) in FIG. 20;

(4) The kidney tissue of rats in the low-dose group exhibits no obvious abnormality; topical renal tubule interstitial connective tissue of rats in the high-dose group is proliferated, and a relatively large amount of fibroblasts are seen, as shown by the black arrow 15 (bottom left) in FIG. 20, which are accompanied with a small amount of infiltrated inflammatory cells, as shown by the red arrow 15 (far right) in FIG. 20, a small amount of slightly dilated renal tubular lumen, as shown by the yellow arrow 15 (second right) in FIG. 20, and a small number of necrotic and apoptotic renal tubular epithelial cells, nuclear pyknosis and deep staining, or fragmentation and dissolution, as shown by the green arrow 15 (top) in FIG. 20; no obvious abnormalities are seen in other tissues.

Rat Tissue Inflammation Score

The tissue inflammation score shows that rats in the low-dose group have no inflammatory response, and rats in the high-dose group have mild inflammatory response in the spleen, lung, and kidney, as shown in Table 3.

TABLE 3
Inflammation score of Rattus norvegicus organs (n=3)

| | | Inflammation score | | |
|---|---|---|---|---|
| | | Control | Low-dose | High-dose |
| Slice No. | Gender | group | group | group |
| Heart | Female | 0 | 0 | 0 |
| | Male | 0 | 0 | 0 |
| Liver | Female | 0 | 0 | 0 |
| | Male | 0 | 0 | 0 |
| Spleen | Female | 0 | 0 | 1 |
| | Male | 0 | 0 | 1 |
| Lung | Female | 0 | 0 | 1 |
| | Male | 0 | 0 | 1 |
| Kidney | Female | 0 | 0 | 0 |
| | Male | 0 | 0 | 1 |

Note:
The inflammation score is based on the overall conditions of the whole piece of slice.

Inflammation degree score: 0 (no or very little inflammation); 1 (mild inflammation); 2 (moderate inflammation); and 3 (severe inflammation).

In this acute toxicity test, no Rattus norvegicus death is observed in both the high-dose group (0.14 g/kg) and the low-dose group (0.07 g/kg), which proves that the lethal dose of hordenine is greater than 0.14 g/kg. Compared with the control group, the rats in the treatment groups show no significant differences in physiological conditions, weight changes, behavioral activities, and the like; pathological slices and biochemical experiments show slight inflammation in the liver, spleen, and kidneys of rats in the high-dose group, which proves that hordenine is less toxic, and can be used under this dose. The maximum dose in this experiment is much larger than the dose for subsequent pharmacodynamic experiments, so hordenine can be used in subsequent experiments at a pharmacodynamic dose.

In summary, one or more embodiments of the present disclosure provide the use of hordenine in the preparation of an inhibitor for inhibiting a MAPK signaling pathway; it has been found that hordenine can help treat prolactinoma and adrenocorticotrophic hormone adenoma and exert drug efficacy by inhibiting the MAPK signaling pathway, solving the dilemma of medication shortage for patients of prolactinoma and adrenocorticotrophic hormone adenoma.

One or more embodiments of the present disclosure also provide a MAPK signaling pathway inhibitor, in which a new substance has been found to inhibit the MAPK signaling pathway, and the substance has a significant inhibitory effect on the MAPK signaling pathway.

In addition, one or more embodiments of the present disclosure also provide the use of a MAPK signaling pathway inhibitor in the manufacture of a medicament for the treatment of prolactinoma and adrenocorticotrophic hormone adenoma, and this use provides a new substance of medicament for the treatment of prolactinoma and adrenocorticotrophic hormone adenoma, which is helpful for the treatment of prolactinoma and adrenocorticotrophic hormone adenoma by inhibiting the MAPK signaling pathway.

The examples as described above are only illustrative examples of the present disclosure, and are not intended to limit the present disclosure. For those skilled in the art, the present disclosure may have various modifications and changes. Any modifications, equivalent substitutions, improvements, and the like made within the spirit and principle of the present disclosure shall be included in the protection scope of the present disclosure.

### INDUSTRIAL APPLICABILITY

The inventor has found that hordenine can help treat prolactinoma and adrenocorticotrophic hormone adenoma by inhibiting TLR4/NF-κB/MAPK signaling pathway, thereby exerting drug efficacy. This solves the dilemma of medication shortage for patients of prolactinoma and adrenocorticotrophic hormone adenoma. The MAPK signaling pathway inhibitors of the present disclosure, especially hordenine, can be used to treat prolactinoma and adrenocorticotrophic hormone adenoma, which is helpful for the treatment of prolactinoma and adrenocorticotrophic hormone adenoma by inhibiting the MAPK signaling pathway.

What is claimed is:

1. Use of hordenine in preparation of an inhibitor for inhibiting a MAPK signaling pathway.

2. The hordenine or a pharmaceutically acceptable salt thereof in the use of claim 1, wherein the MAPK signaling pathway is TLR4/NF-κB/MAPK signaling pathway.

3. The hordenine or a pharmaceutically acceptable salt thereof in the use of claim 1, wherein the inhibition of a MAPK signaling pathway is used for inhibiting the expression of at least one selected from the group consisting of PRL, ATCH, MAPK12, TLR4, IL-β and TNF-α.

4. A MAPK signaling pathway inhibitor, comprising hordenine.

5. The MAPK signaling pathway inhibitor of claim 4, wherein the MAPK signaling pathway is TLR4/NF-κB/MAPK signaling pathway;
wherein the MAPK signaling pathway inhibitor is used for inhibiting the expression of at least one selected from the group consisting of MAPK12, TLR4, PRL, ACTH, IL-β, and TNF-α.

6. Use of the MAPK signaling pathway inhibitor of claim 4 in manufacturing a medicament for treatment of pituitary tumors, the pituitary tumors comprising prolactinoma and adrenocorticotrophic hormone adenoma.

7. A method for treating pituitary tumors, comprising administering a MAPK signaling pathway inhibitor to a subject in need.

8. The method of claim 7, wherein the pituitary tumors comprise prolactinoma and adrenocorticotrophic hormone adenoma.

9. The method of claim 7, wherein the MAPK signaling pathway inhibitor comprises hordenine.

10. Use of the MAPK signaling pathway inhibitor of claim 4 in the manufacture of a medicament for treatment of a disease associated with MAPK signaling pathway abnormality;
wherein the MAPK signaling pathway abnormality is a TLR4/NF-κB/MAPK signaling pathway abnormality, and comprises the overexpression of at least one selected from the group consisting of MAPK12, TLR4, PRL, ACTH, IL-β, and TNF-α.

11. A method for treating a disease associated with MAPK signaling pathway abnormality, comprising administering the MAPK signaling pathway inhibitor of claim 4 to a subject in need.

12. Use of the MAPK signaling pathway inhibitor of claim 4 in the treatment of a disease associated with MAPK signaling pathway abnormality.

13. The MAPK signaling pathway inhibitor in the use of claim 12, wherein the MAPK signaling pathway abnormality is TLR4/NF-κB/MAPK signaling pathway abnormality.

14. The MAPK signaling pathway inhibitor in the use of claim 12, wherein the MAPK signaling pathway abnormality comprises the overexpression of at least one selected from the group consisting of MAPK12, TLR4, PRL, ACTH, IL-β, and TNF-α.
